(19) United States
(12) Patent Application Publication
(10) Pub. No.: US 2008/0183181 A1
(43) Pub. Date: Jul. 31, 2008

(54) Enclosed Protective Packaging

(75) Inventors: Kevin Treacy, Athenry (IE); Thomas Farrell, Claregalway (IE); Patricia O'beirne, Bohermore (IE); Kevin Boyle, Renmore (IE); John Clifford, Barna (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/668,657

(22) Filed: Jan. 30, 2007

(51) Int. Cl. A61F 2/00

(52) U.S. Cl. 606/108

(57) ABSTRACT

A vascular treatment device delivery system includes an elongated protective sheath having a distal end and a proximal end, a delivery catheter disposed within an axial lumen of the elongated protective sheath, and a luer fitting operably connected to a proximal end of the delivery catheter. The system further includes a sealable container attached to the proximal end of the protective sheath, the sealable container surrounding the luer fitting and a sterility barrier attached to a portion of the sealable container.

### FIELD OF THE INVENTION

The invention relates generally to packaging medical devices, and specifically to packaging intravascular devices.

### BACKGROUND OF THE INVENTION

Coronary artery disease (CAD) results from arteriosclerosis of blood vessels serving the heart. Arteriosclerosis is a hardening and narrowing of the arteries commonly accompanied by a deposition of waxy substance therein. This substance, known as plaque, is made of cholesterol, fatty compounds, calcium, and the blood-clotting material fibrin. Often the arteries of the heart can suddenly become so severely blocked that there is an inadequate blood supply after the blockage, leading to the occurrence of a myocardial infarction or “heart attack.”

Soon after a myocardial infarction, the area of cardiac tissue downstream of the blockage may suffer damage. Unless the blockage is resolved relatively quickly, the ischemic cells may begin to die. Often, a surgical procedure, such as a Percutaneous Transluminal Coronary Angioplasty (PTCA) procedure, oftentimes accompanied by stenting of the blocked vessel, is performed to reopen the vessel and maintain blood flow.

Plain-old-balloon-angioplasty (POBA) is an exemplary medical procedure to widen obstructed blood vessels narrowed by plaque deposits. The procedure may be used in coronary or peripheral arteries. In an angioplasty procedure, a catheter having a special inflatable balloon on its distal end is navigated through the patient's arteries and is advanced through the artery to be treated to position the balloon within the narrowed region (stenosis). The region of the stenosis is expanded by inflating the balloon under pressure to forcibly widen the artery. After the artery has been widened, the balloon is deflated and the catheter is removed from the patient.

A significant difficulty associated with balloon angioplasty is that in a considerable number of cases the artery may again become obstructed in the same region where the balloon angioplasty had been performed. The repeat obstruction of the artery (restenosis) may be immediate or, more commonly, may occur at a later time and may require repeat balloon angioplasty.

To reduce the incidence of restenosis, several strategies have been developed. Implantable devices, such as stents, have been used to reduce the rate of angioplasty related restenosis by about half. The stent is placed inside the blood vessel after the angioplasty has been performed. A catheter typically is used to deliver the stent to the arterial site to be treated. The stent may further include one or more therapeutic substance(s) impregnated or coated thereon to limit re-obstruction and/or restenosis.

Implantable devices, such as stents, and the catheters used to deliver the stents to the site of implantation require sterilization prior to implantation. Generally, the catheter and implantable device are packaged and sealed in a protective device prior to sterilization. The packaging, properly sealed, will maintain the sterility of the product until it is withdrawn from the packaging for use. Packaging delivery catheters used for delivery stents or balloons to a treatment site generally requires placing the device in a protective tray or hoop which is then inserted into a pouch and sealed prior to sterilization. However, while packaging medical devices in this manner is generally an effective method of packaging, this method produces an inordinate amount of waste material due to the single use nature of the packaging.

Accordingly, it would be desirable to provide a method of packaging medical devices that would overcome these and other disadvantages.

### SUMMARY OF THE INVENTION

One aspect of the present invention provides a vascular treatment device delivery system. The system comprises an elongated protective sheath having a distal end and a proximal end, a delivery catheter disposed within an axial lumen of the elongated protective sheath, and a luer fitting operably connected to a proximal end of the delivery catheter. The system further includes a sealable container attached to the proximal end of the protective sheath, the sealable container surrounding the luer fitting and a sterility barrier attached to a portion of the sealable container.

Another aspect of the present invention provides a method of packaging a vascular treatment device delivery system. The method comprises providing an elongate protective sheath and a sealable container, the sealable container attached to a proximal end of the protective sheath and disposing a medical device having a luer fitting at a proximal end within a lumen of the protective sheath and a chamber of the sealable container. The method further comprises attaching a sterility barrier to the sealable container; sealing a distal end of the protective sheath; and sterilizing the sealed medical device.

Another aspect of the present invention provides a vascular treatment device delivery system. The system comprises an elongated protective sheath, a luer fitting in communication with a distal end of the elongated protective sheath, container means for containing the luer fitting and means for sealing the containing means to enclose the luer fitting.

The foregoing, and other, features and advantages of the invention will become further apparent from the following detailed description of the presently preferred embodiments, read in conjunction with the accompanying drawings. The drawings are not drawn to scale. The detailed description and drawings are merely illustrative of the invention rather than limiting, the scope of the invention being defined by the appended claims in equivalence thereof.

### BRIEF DESCRIPTION OF THE DRAWINGS

FIG. 1 is a schematic view of one embodiment of a vascular treatment device delivery system with protective packaging in accordance with the present invention;

FIG. 2 is a schematic view of another embodiment of a vascular treatment device delivery system with protective packaging in accordance with the present invention;

FIGS. 3 and 4 are schematic views of another embodiment of a vascular treatment device delivery system with protective packaging in accordance with the present invention; and

FIG. 5 is a flow chart of one embodiment of a method of packaging a vascular treatment device delivery system accordance with the present invention.

### DETAILED DESCRIPTION OF THE PRESENTLY PREFERRED EMBODIMENTS

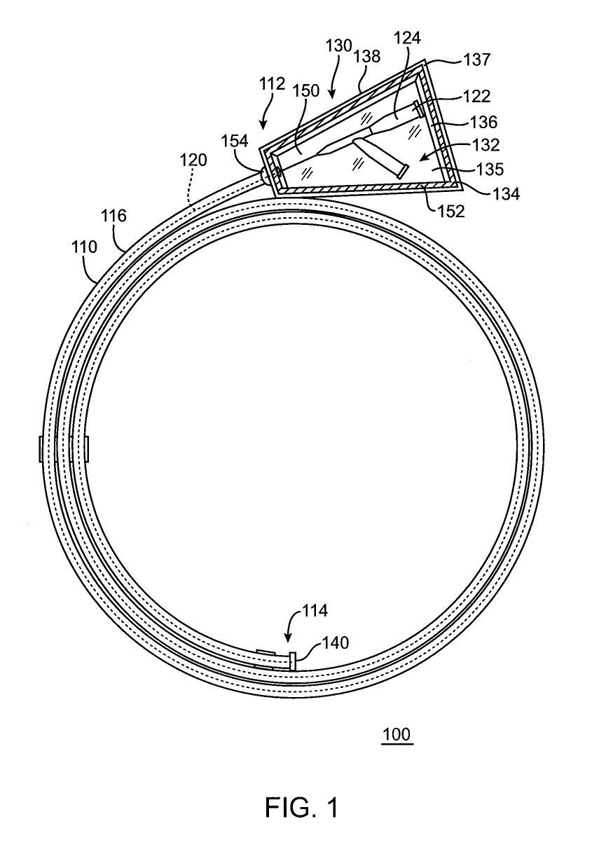

FIG. 1 shows one embodiment of a vascular treatment device delivery system with protective packaging in accordance with the present invention at 100. System 100 includes an elongated protective sheath 110, a delivery catheter 120, a sealable container 130, a distal end cap, 140 and a sterility barrier 150.

Protective sheath 110 comprises an elongate tubular member have a proximal end 112, a distal end 114 and a central lumen 116. Protective sheath 110 is sized to receive an elongate medical device such as, for example, delivery catheter 120. In one embodiment, protective sheath comprises a looped coil as are known in the art. In another embodiment, protective sheath 110 comprises a linear tubular member for receiving elongate medical devices that are packaged in a substantially straight configuration. Protective sheath 110 may be composed of any polymeric material suitable for packaging medical devices and maintaining sterility. In one embodiment, protective sheath 110 is composed of an extruded high-density polyethylene (HDPE).

Delivery catheter 120 is removably disposed in lumen 116. Delivery catheters for delivering vascular treatment devices are well known in the art. The delivery catheter components may be formed of conventional materials used in angioplasty and stent delivery catheters and may be formed in conventional manners well known in the art. In one embodiment, delivery catheter 120 comprises a stent delivery catheter. The stent delivery catheter may be, for example, a rapid exchange design or an over-the-wire design, as are known in the art. Delivery catheter 120 comprises an elongate member having a distal end for delivering the treatment device such as a stent or an angioplasty balloon and a proximal end 122. In one embodiment, proximal end 122 comprises a Luer fitting 124 as are well known in the art. Proximal end 122 may comprise other types of fittings such as, for example, single lumen rapid exchange luer or double lumen over-the-wire luer

System 100 further includes a sealable container 130 attached to the proximal end 112 of protective sheath 110. Sealable container 130 is sized and shaped to receive luer fitting 124. In one embodiment, the sealable container 130 surrounds the luer fitting 124 attached to the proximal end 122 of the delivery catheter 120 and the remainder of the delivery catheter distal to the luer fitting is disposed within the lumen 116 of the protective sheath 110.

In one embodiment, sealable container 130 comprises a tray 138 composed of a bottom portion 135 and a plurality of walls 136 that form a chamber 132 for receiving the luer fitting 124. Tray 138 further comprises a flange portion 134 extending substantially perpendicular to an upper portion of the walls 136. The flange portion 134 includes an upper surface 137 for receiving a perimeter portion 152 of sterility barrier 150, discussed in more detail below.

Tray 138 may be composed of the same or similar material as protective sheath 110. In one embodiment, tray 138 is composed of a polymer such as HDPE. Tray 138 may be made, for example, of a high barrier plastic, or other suitable materials. Tray 138 may be thermoformed, for example, from polyethylene, PVC, polystyrene, polypropylene, polycarbonate or another suitable material. Tray 138 fixedly attached to the proximal end 112 of protective sheath 110 such that the sheath lumen 116 is in fluid communication with chamber 132. In one embodiment, tray 138 is attached to protective sheath 110 by welding. In another embodiment, tray 138 is attached to protective sheath 110 by adhesive. Tray 138 may be attached to protective sheath 110 by any method known in the art that will maintain the sterility of the enclosed medical device.

A sterility barrier 150 is placed over and sealed to tray 138 after the delivery catheter 120 and luer fitting 124 are placed within the protective sheath 110 and tray 138, respectively, prior to sterilization. Sterility barrier 150 comprises any material suitable for permitting passage of the small molecule sterilizing gas to the interior of the protective sheath 110 and sealable container 130 while remaining a barrier to liquids, bacteria, viruses, and other larger substances which compromise sterility of the enclosed medical device. Suitable gas permeable materials include high-density polyethylene (HDPE), typically a spun HDPE or other spun olefin. In one embodiment, the sterility barrier comprises a spun HDPE material, such as, for example, Tyvek® microbial barrier material, e.g., Tyvek® 1073B, Tyvek® 2FS and Tyvek® 1059B) from E. I DuPont de Nemours and Company.

Sterility barrier 150 material is sealed to the flange portion 134 of tray 138 by heat sealing or adhesive. In one embodiment, the perimeter portion 152 of sterility barrier 150 is coated with an adhesive for sealing the sterility barrier material to the flange surface 137.

In one embodiment, enclosure of the medical device 120 within the protective sheath 110 is completed by placing an end cap 140 at the distal end 114 of the protective sheath 110. In one embodiment, end cap 140 comprises a cap including a sterility barrier material, such as, spun HDPE material, such as, for example, Tyvek® microbial barrier material, discussed above. In another embodiment, end cap 140 comprises a plug composed of HDPE. In yet another embodiment, end cap 140 comprises a crimped and sealed end of the distal end 114 of protective sheath 110. In the preferred embodiment, end cap 140 allows for the penetration of the sterilizing gas and provides a barrier to liquids, bacteria, viruses, and other larger substances which compromise sterility of the enclosed medical device.

Sterilization of the enclosed medical device, for example, delivery catheter 120, is then effected by placing the sealed package in an environment including a sterilant gas, such as ethylene oxide (EtO). The permeability required for the sterilizing gas to permeate into the interior of the protective sheath 110 and sealable container 130 contact the medical device, can vary depending upon the pressure differential, the temperature and the time available for permeation of the sterilizing gas into the interior.

Sterility barrier 150 may include a pull tab 154 for removing sterilization barrier 150 prior to use of the medical device. In another embodiment, system 100 may also include desiccants, and oxygen absorbers within chamber 132 of the sealable container 130. Oxygen absorbers can be placed in fluid communication with the interior of the package, such as within a permeable packet that is placed within the chamber of the sealable container adjacent the luer fitting.

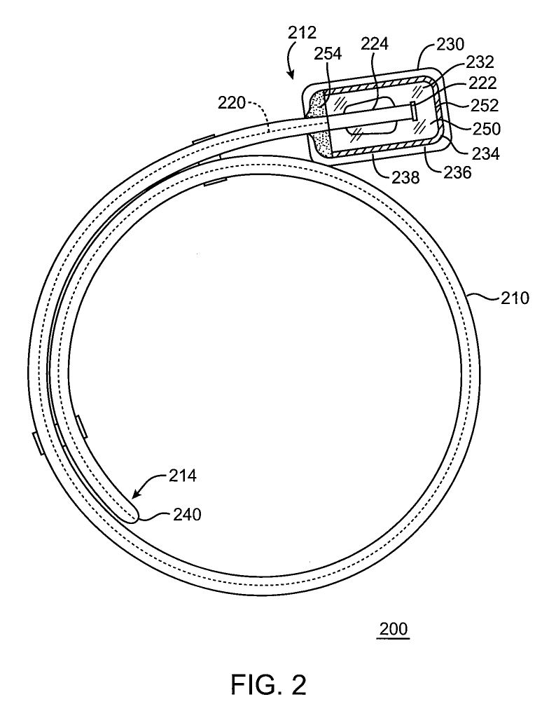

FIG. 2 illustrates another embodiment of a vascular treatment device delivery system with protective packaging in accordance with the present invention, at 200.

System 200 includes an elongated protective sheath 210, a delivery catheter 220, a sealable container 230, a distal end cap, 240 and a sterility barrier 250.

In one embodiment, protective sheath 210 and delivery catheter 220 are the same as, or similar to protective sheath 110 and delivery catheter 120, described above.

System 200 further includes a sealable container 230 disposed at the proximal end 212 of protective sheath 210. Sealable container 230 is sized and shaped to receive luer fitting 224. In one embodiment, sealable container 230 comprises a molded retainer 238 composed of a wall 236 that forms a chamber 232 for receiving luer fitting 224. Molded retainer 238 includes an outer surface 236 for receiving a perimeter portion 252 of sterility barrier 250. In one embodiment, molded retainer 238 comprises a molded container fixedly attached to the proximal end of protective sheath 210. In another embodiment, molded retainer 238 and protective sheath 210 are formed as a unitary piece. In one embodiment, molded retainer 238 and protective sheath 210 are composed of extruded HDPE.

A sterility barrier 250 is placed over and sealed to molded retainer 238 after the delivery catheter 220 and luer fitting 224 are placed within the protective sheath 210 and molded retainer 238, respectively, prior to sterilization. Sterility barrier 250 comprises any material suitable for permitting passage of the small molecule sterilizing gas to the interior of the protective sheath 210 and sealable container 230 while remaining a barrier to liquids, bacteria, viruses, and other larger substances which compromise sterility of the enclosed medical device. Sterility barrier 250 may be the same as, or similar to sterility barrier 150, described above.

Sterility barrier 250 material is sealed to the outer surface 236 of molded retainer 238 by heat sealing or adhesive. In one embodiment, the perimeter portion 252 of sterility barrier 250 is coated with an adhesive for sealing the sterility barrier material to the outer surface 236.

Sterility barrier 250 may include a pull tab 254 for removing sterilization barrier 250 prior to use of the medical device. In another embodiment, system 200 may also include dessicants, and O₂ absorbers within chamber 232 of the sealable container 230.

In one embodiment, enclosure of the medical device 220 within the protective sheath 210 is completed by placing an end cap 240 at the distal end 214 of the protective sheath 210. In one embodiment, end cap 240 comprises a cap including a sterility barrier material, such as, spun HDPE material, such as, for example, Tyvek® microbial barrier material, discussed above. In another embodiment, end cap 240 comprises a plug composed of HDPE. In yet another embodiment, end cap 240 comprises a crimped and sealed end of the distal end 214 of protective sheath 210. In the preferred embodiment, end cap 240 allows for the penetration of the sterilizing gas and provides a barrier to liquids, bacteria, viruses, and other larger substances which compromise sterility of the enclosed medical device.

Sterilization of the enclosed medical device, for example, delivery catheter 220, is then effected by placing the sealed package in an environment including a sterilant gas, such as ethylene oxide (EtO).

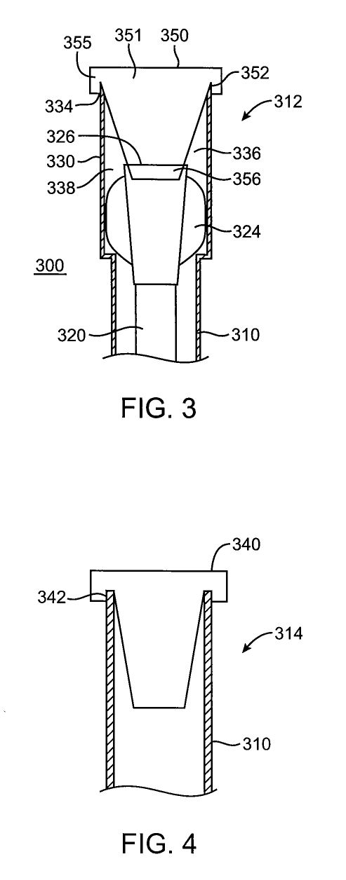

FIGS. 3 and 4 are detailed views of another embodiment of a vascular treatment device delivery system with protective packaging in accordance with the present invention, at 300. FIG. 3 illustrates a proximal end 312 of system 300 and FIG. 4 illustrates a distal end 314 of system 300.

System 300 includes an elongated protective sheath 310, a delivery catheter 320, a sealable container 330, a distal end cap, 340 and a sterility barrier 350.

In one embodiment, protective sheath 310 and delivery catheter 320 are the same as, or similar to protective sheath 110 and delivery catheter 120, described above.

System 300 further includes a sealable container 330 disposed at the proximal end 312 of protective sheath 310. Sealable container 330 is sized and shaped to receive luer fitting 324. Sealable container 330 comprises an axial luer chamber 338 comprising an enlarged lumen 336 extending from proximal end 312 of protective sheath 310. Luer chamber 338 is sized for receiving luer fitting 324 and sterility barrier 350. Luer chamber 338 includes an end portion 334 for receiving a sealing member 352 of sterility barrier 350. In one embodiment, luer chamber 338 and protective sheath 310 are formed as a unitary piece. In one embodiment, luer chamber 338 and protective sheath 310 are composed of extruded HDPE.

A sterility barrier 350 is attached and sealed to luer chamber 338 after the delivery catheter 320 and luer fitting 324 are placed within the protective sheath 310 and luer chamber 338, respectively, prior to sterilization. Sterility barrier 350 comprises a plug composed of any material suitable for permitting passage of the small molecule sterilizing gas to the interior of the protective sheath 310 and sealable container 130 while remaining a barrier to liquids, bacteria, viruses, and other larger substances which compromise sterility of the enclosed medical device. In one embodiment, sterility barrier 350 comprises a plug 351 composed of polytetrafluoroethylene (PTFE).

Plug 351 includes a cap portion 355 and a luer contacting portion 356 disposed opposite of cap portion 355. Cap portion 355 includes a sealing portion 352 for contacting end portion 334 of luer chamber 338 in a sealing engagement. In one embodiment, the sealing engagement comprises an interference fit. In place, sealing portion 352 and end portion 334 form a seal, providing a sterility barrier. Luer contacting portion 356 is sized to fit within the end opening 326 of luer 324. In place, luer contacting portion 356 secures luer fitting 324 within luer chamber 338.

In one embodiment, enclosure of the medical device 320 within the protective sheath 310 is completed by placing an end cap 340 at the distal end 314 of the protective sheath 310 (see FIG. 4). In one embodiment, end cap 340 is composed of polytetrafluoroethylene (PTFE). In the preferred embodiment, end cap 340 allows for the penetration of the sterilizing gas and provides a barrier to liquids, bacteria, viruses, and other larger substances which compromise sterility of the enclosed medical device.

Sterilization of the enclosed medical device, for example, delivery catheter 320, is then effected by placing the sealed package in an environment including a sterilant gas, such as ethylene oxide (EtO).

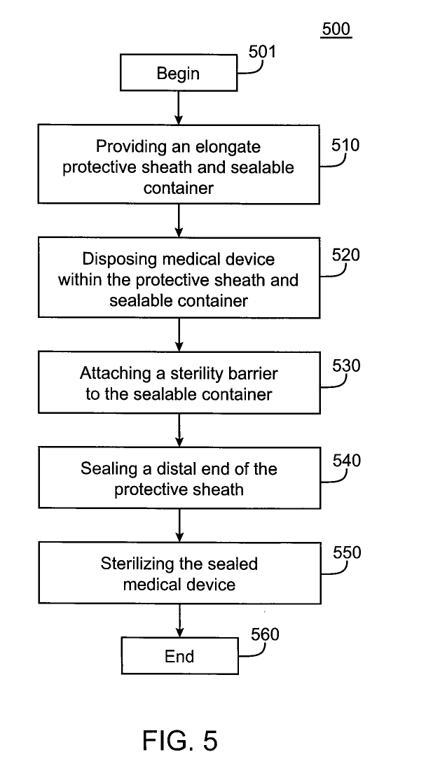

FIG. 5 is a flow chart of one embodiment of a method 500 for packaging a vascular treatment device delivery system. Method 500 begins at step 501.

At step 510, an elongate protective sheath including a sealable container attached to a proximal end of the protective sheath is provided. The sealable container may be sealable container 130, 230 or 330 described above. Next, a medical device having a proximal luer fitting is disposed within the protective sheath and sealable container, the luer fitting being disposed within the sealable container, step 520. In one embodiment the medical device comprises a vascular treatment device and delivery system.

At step 530, a sterility barrier is attached to a portion of the sealable container to enclose the luer fitting within the sealable container. In one embodiment, the sterility barrier comprises a microbial barrier material that is heat sealed to a surface of the sealable container. In another embodiment, the sterility barrier is attached to a surface of the sealable container using adhesive. In another embodiment, the sterility barrier comprises a plug that is inserted into an opening of the sealable container.

At step 540, a distal end of the proetective sheath is sealed. In one embodiment an end plug is placed at a distal end of the protective sheath to seal the distal end of the protective sheath and to provide a sterility barrier. Next, at step 550, the sealed vascular treatment device delivery system is sterilized. Sterilizing the vascular treatment device delivery system comprises placing the sealed package in an environment including a sterilant gas, such as ethylene oxide (EtO). Method 500 ends at 560.

Those with skill in the art will recognize that the packaging of medical devices in accordance with the present invention has many benefits. For example, packaging of medical devices in accordance with the present invention will reduce the amount of waste material as well as costs associated with this reduction of waste material. Further reductions in cost may be afforded in packaging several enclosed and sterilized devices within a single shipping and storage carton. Cost savings may also be realized by the reduction in the amount of time spent in packaging the devices for sterilization and shipping.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A vascular treatment device delivery system, comprising:
an elongated protective sheath having a distal end and a proximal end;
a delivery catheter disposed within an axial lumen of the elongated protective sheath;
a luer fitting operably connected to a proximal end of the delivery catheter;
a sealable container attached to the proximal end of the protective sheath, the sealable container surrounding the luer fitting; and
a sterility barrier attached to a portion of the sealable container.

2. The system of claim 1, further comprising:
an end cap disposed at a distal end of the protective sheath.

3. The system of claim 1, wherein the sealable container comprises a tray, the tray comprising:
a bottom portion;
a plurality of walls extending from the bottom portion, the bottom portion and plurality of walls forming a chamber; and
a flange portion extending from an upper portion of the plurality of the plurality of walls, the flange portion having an upper surface for receiving a perimeter portion of the sterility barrier.

4. The system of claim 3, wherein the sterility barrier comprises a gas permeable material.

5. The system of claim 1 wherein the sealable container comprises a molded retainer, the molded retainer comprising a wall, the wall forming a chamber for receiving a luer fitting, and the wall including an outer surface for receiving a perimeter portion of the sterility barrier.

6. The system of claim 5 wherein the molded retainer and the protective sheath are formed as a unitary piece.

7. The system of claim 1 wherein the sealable container comprises an axial luer chamber, the axial luer chamber comprising an enlarged lumen extending from the proximal end of the protective sheath, wherein the axial luer chamber is sized to receive the luer fitting and a portion of a sterility barrier.

8. The system of claim 7 wherein the sterility barrier comprises a plug, the plug including an cap portion and a luer contacting portion, the luer contacting portion disposed opposite of the cap portion.

9. The system of claim 8 wherein the plug is composed of a polytetrafluoroethylene material.

10. The system of claim 8 wherein the luer contacting portion is sized to fit within an end opening of the luer fitting.

11. The system of claim 8 wherein the cap portion includes a sealing portion for sealingly mating with a proximal end portion of the protective sheath.

12. The system of claim 8 further comprising a distal end cap disposed at a distal end of the protective sheath.

13. The system of claim 12 wherein the distal end cap is composed of a polytetrafluoroethylene material.

14. The system of claim 1, wherein at least a portion of the sterility barrier is heat-sealed to a surface of the sealable container.

15. The system of claim 1, wherein at least a portion of the sterility barrier is sealed to a surface of the sealable container by adhesive.

16. The system of claim 1 further comprising a pull tab operably connected to a portion of the sterility barrier.

17. The system of claim 2 wherein the end cap comprises a gas permeable material.

18. A method of packaging a vascular treatment device delivery system, the method comprising:
providing an elongate protective sheath and a sealable container, the sealable container attached to a proximal end of the protective sheath;
disposing a medical device having a luer fitting at a proximal end within a lumen of the protective sheath and a chamber of the sealable container;
attaching a sterility barrier to the sealable container;
sealing a distal end of the protective sheath; and
sterilizing the sealed medical device.

19. A vascular treatment device delivery system, comprising:
an elongated protective sheath;
a luer fitting in communication with a distal end of the elongated protective sheath;
container means for containing the luer fitting; and
means for sealing the containing means to enclose the luer fitting.
